(19) United States
(12) Patent Application Publication
(10) Pub. No.: US 2023/0097410 A1
(43) Pub. Date: Mar. 30, 2023

(54) WOUND DRESSINGS WITH ACID-INDUCED GROWTH FACTOR RELEASE

(71) Applicants: KCI Licensing, Inc., San Antonio, TX (US); Systagenix Wound Management, Limited, West Sussex (GB)

(72) Inventors: Prathamesh Madhav KHARKAR, San Antonio, TX (US); Carrina WARD, Gargrave (GB); Katie BOURDILLON, Leeds (GB); Alexander WAITE, Cowling (GB)

(21) Appl. No.: 17/790,936

(22) PCT Filed: Jan. 7, 2021
(86) PCT No.: PCT/IB2021/050098

§ 371 Date: Jul. 5, 2022

Related U.S. Application Data
(60) Provisional application No. 62/958,047, filed on Jan. 7, 2020.

(51) Int. Cl. A61L 26/00

(52) U.S. Cl. CPC A61L 26/0066; A61L 26/0033; A61L 26/0023; A61L 26/0085; A61L 2300/414; A61L 2300/252

(57) ABSTRACT

The present technology generally relates to a biomaterial that includes a thiolated biopolymer and one or more agents that include growth factors, peptides, or combinations thereof, where the one or more agents are conjugated to the thiolated biopolymer. Also disclosed herein are wound dressings that include the biomaterial of the present technology, methods of treating a wound upon application of the wound dressing, and kits including the wound dressing and instructions for use.

### CROSS-REFERENCE TO RELATED APPLICATIONS

This application claims the benefit of priority to U.S. Provisional Application No. 62/958,047, filed on Jan. 7, 2020, which is incorporated herein by reference in its entirety.

### TECHNICAL FIELD

The present technology relates generally to biomaterials and wound dressings that include the biomaterial for facilitating wound healing. In particular, the present technology relates to biomaterials that include biopolymers chemically-modified to include growth factors and/or peptides which release upon exposure to acidic wound environments.

### BACKGROUND

The following description of the background of the present technology is provided simply as an aid in understanding the present technology and is not admitted to describe or constitute prior art to the present technology.

A wide variety of materials and devises, generally characterized as “dressings,” are generally known in the art for use in treating an injury or other disruption of tissue. Such wounds may be the result of trauma, surgery, or disease, and may affect skin or other tissues. Wound healing may be stalled due to a lack of any biological growth factors or other agents suitable for improving and/or accelerating the progression of wound healing.

### SUMMARY

In one aspect, the present technology provides a biomaterial that includes a thiolated biopolymer and one or more agents that include growth factors, peptides, or combinations thereof, where the one or more agents are conjugated to the thiolated biopolymer.

In another related aspect, the present technology provides a method for preparing a biomaterial as disclosed herein in any embodiment. The method includes reacting a biopolymer with a thiolactone to obtain a thiolated biopolymer, reacting the thiolated biopolymer with one or more agents selected from growth factors, peptides, or combinations thereof to obtain the biomaterial, wherein the one or more agents are conjugated to the thiolated biopolymer via a disulfide linkage.

In a related aspect, the present technology provides a wound dressing that includes the biomaterial as disclosed herein in any embodiment.

In an aspect, methods for treating a wound in a subject in need thereof are provided, wherein the method includes administering to the wound a wound dressing that includes the biomaterial of any embodiment disclosed herein.

In an aspect, methods for treating a wound in a subject in need thereof are provided, wherein the method includes administering to the wound a wound dressing that includes the biomaterial of any embodiment disclosed herein.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

It is to be appreciated that certain aspects, modes, embodiments, variations and features of the present technology are described below in various levels of detail in order to provide a substantial understanding of the present technology.

### Definitions

The definitions of certain terms as used in this specification are provided below. Unless defined otherwise, all technical and scientific terms used herein generally have the same meaning as commonly understood by one of ordinary skill in the art to which this present technology belongs.

The following terms are used throughout as defined below.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would mean “9 wt. % to 11 wt. %.” It is to be understood that when “about” precedes a term, the term is to be construed as disclosing “about” the term as well as term without modification by “about”—for example, “about 10 wt. %” discloses “9 wt. % to 11 wt. %” as well as discloses “10 wt. %.”

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “mammalian recombinant collagen” refers to collagen manufactured by culturing a non-human organism or mammalian or non-mammalian cells to express at least one exogenous gene encoding a collagen in the culturing system. The term “human recombinant collagen” refers to collagen manufactured by culturing a non-human organism or mammalian or non-mammalian cells to express at least one human gene encoding a collagen. The human recombinant collagen may be selected from the group consisting of collagen type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XXIII, type XXIV, type XXV, type XXVI, type XXVII, and mixtures of any two or more thereof. The human recombinant collagen can be collagen of one type free of any other type, or can be a mixture of collagen types. Suitably, the human recombinant collagen includes collagens selected from collagen type I, collagen type II, or mixtures thereof. The term “bovine recombinant collagen” refers to collagen manufactured by culturing a non-human organism or mammalian or non-mammalian cells to express at least one bovine gene encoding a collagen. The bovine recombinant collagen may be selected from collagen type I, type II, type III, type IV, or mixtures of any two or more thereof. The bovine recombinant collagen can be collagen of one type free of any other type, or can be a mixture of collagen types. Suitably, the bovine recombinant collagen includes collagens selected from collagen type I, collagen type III, and mixtures thereof.

As understood by one of ordinary skill in the art, “molecular weight” (also known as “relative molar mass”) is a dimensionless quantity but is converted to molar mass by multiplying by 1 gram/mole—for example, collagen with a weight-average molecular weight of 5,000 has a weight-average molar mass of 5,000 g/mol.

As used herein, the “administration” of a wound dressing to a subject includes any route of introducing or delivering to a subject a diagnostic wound dressing to perform its intended function. Administration can be carried out by any suitable route, including but not limited to, topical administration. Administration includes self-administration and the administration by another.

As used herein, the term “effective amount” refers to a quantity sufficient to achieve a desired therapeutic and/or prophylactic effect, e.g., an amount which results in the decrease in a wound described herein or one or more signs or symptoms associated with a wound described herein. In the context of therapeutic or prophylactic applications, the amount of a composition administered to the subject will vary depending on the composition, the degree, type, and severity of the wound and on the characteristics of the individual. The compositions can also be administered in combination with one or more additional therapeutic compounds. In the methods described herein, the therapeutic compositions may be administered to a subject having one or more wounds.

As used herein, the terms “individual”, “patient”, or “subject” can be an individual or organism, a vertebrate, a mammal, or a human. In some embodiments, the individual, patient, or subject is a human.

“Treating” or “treatment” as used herein covers the treatment of a wound described herein, in a subject, such as a human, and includes: (i) inhibiting a wound, i.e., arresting its development; (ii) relieving a wound, i.e., causing regression of the wound; (iii) slowing progression of the wound; and/or (iv) inhibiting, relieving, or slowing progression of one or more symptoms of the wound. In some embodiments, treatment means that the symptoms associated with the wound are, e.g., alleviated, reduced, cured, or placed in a state of remission.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound and/or can include one or more administrations for the treatment of an acute wound.

### The Wound Dressing of the Present Technology

While being effective, current wound dressings, such as collagen based wound dressings, struggle due to a lack of any biological growth factors within them.

The present disclosure is directed to wound dressings that include a biomaterial, where the biomaterial includes a thiolated biopolymer and one or more agents selected from growth factors, peptides, or combinations thereof. The one or more agents are conjugated to the thiolated biopolymer. The wound dressing of the present technology advantageously releases the one or more agents (e.g., growth factors and/or peptides) upon exposure to the acidic environment often found in wounds.

Thus, in an aspect, a biomaterial is provided that includes a thiolated biopolymer and one or more agents that include growth factors, peptides, or combinations thereof, where the one or more agents are conjugated to the thiolated biopolymer. In another aspect, a wound dressing is provided that includes the biomaterial as described herein in any embodiment. The wound dressing may be in the form of a foam.

### Biomaterial

In one aspect, the present technology provides a biomaterial that includes a thiolated biopolymer and one or more agents that include growth factors, peptides, or combinations thereof, where the one or more agents are conjugated to the thiolated biopolymer.

As used herein, the term “thiolated biopolymer” refers to a biopolymer as disclosed herein in any embodiment that has been chemically-modified to incorporate thiol (—SH) functional groups. For example, in any embodiment disclosed herein, the thiolated biopolymer may be obtained by reaction of a substituted or unsubstituted thiolactone with a primary amine group of the biopolymer, where the thiolactone may be a 3- to 12-member ring having 2 to 10 carbons. In any embodiment disclosed herein, the thiolactone may be 3- to 8-membered ring having 2 to 6 carbons. In any embodiment disclosed herein, the thiolactone may be represented by the following structure:

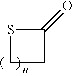

where n may be 0, 1, 2, 3, 4, 5, 6, 7, 8, or 9, and where the thiolactone is optionally substituted with an amide group. In any embodiment disclosed herein, n may be 2. In any embodiment disclosed herein, the thiolactone may be substituted with —NHC(O)CH₃. In any embodiment disclosed herein, the thiolactone may be N-acetyl homocysteine thiolactone.

In any embodiment described herein, the biomaterial may include from about 0.01 weight percent (wt. %) to 99 wt. % of the thiolated biopolymer based on the total weight of the biomaterial. For example, in any embodiment described herein, the amount of thiolated biopolymer may be about 0.01 wt. %, about 0.1 wt. %, about 0.5 wt. %, about 1 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 91 wt. %, about 92 wt. %, about 93 wt. %, about 94 wt. %, about 95 wt. %, about 99 wt. %, or any value including and/or in between any two of the preceding values based on the total weight of the biomaterial. Suitable ranges may include, but are not limited to, from about 0.01 wt. % to about 99 wt. %, about 1 wt. % to about 99 wt. %, about 30 wt. % to about 95 wt. %, about 30 wt. % to about 70 wt. %, about 35 wt. % to about 75 wt. %, or any value including and/or in between any two of the preceding values.

The thiolated biopolymer of the biomaterial may be a bio-resorbable biopolymer that has been chemically-modified to incorporate thiol functional groups. In any embodiment disclosed herein, the bio-resorbable biopolymer may include, but is not limited to, collagen, oxidized cellulose, a polysaccharide, chitosan, gelatin, hyaluronic acid, elastin, fibronectin, or a combination of nay two or more thereof. The oxidized cellulose may be an oxidized regenerated cellulose (ORC) of any embodiment disclosed herein. In any embodiment disclosed herein, the thiolated biopolymer may include a thiolated collagen, thiolated cellulose, or a mixture thereof.

In any embodiment disclosed herein, the thiolated collagen may be a collagen thiolated as disclosed herein. The collagen may be recombinant or naturally occurring. In any embodiment disclosed herein, the collagen may include mammalian collagen. For example, in any embodiment described herein, the mammalian collagen may include recombinant or naturally occurring bovine collagen, human collagen, or a combination thereof. In any embodiment herein, the collagen may be a naturally occurring collagen, including but not limited to, type I human collagen, type III human collagen, type I bovine collagen, type III bovine collagen, or a combination of any two or more thereof. In any embodiment herein, the collagen may be recombinant collagen, including but not limited to, type I human recombinant collagen, type III human recombinant collagen, type I bovine recombinant collagen, type III bovine recombinant collagen, or a combination of any two or more thereof.

In any embodiment disclosed herein, mammalian recombinant collagen may be provided by any suitable method known in the art. Additionally or alternatively, in some embodiments, human recombinant collagen may be provided by any suitable method known in the art. For example, the step of providing human recombinant collagen may include following the protocol described in U.S. Pat. No. 5,962,648, the entire content of which is incorporated herein by reference. Further recombinant processes are set forth in U.S. Pat. No. 5,593,859 and WO2004/078129 which are also incorporated herein by references. Additional or alternatively, in some embodiments, collagen will be recombinantly manufactured by culturing a cell which has been transfected with at least one gene encoding a polypeptide including collagen and genes encoding oxidized cellulose and subunits of the post-translational enzyme prolyl 4-hydroxylase, and purifying the resultant collagen monomer therefrom. The human recombinant collagen solution may be subsequently subjected to polymerization or cross-linking conditions to produce an insoluble fibrous collagen. In any embodiment disclosed herein, the biomaterial may include a weight ration of human collagen type I to human collagen type III of about 100:0, about 90:10, about 80:20, about 70:30, about 60:40, about 50:50, about 40:60, about 30:70, about 20:80, about 10:90, about 0:100, or any range including and/or in between any two of the preceding values. The ratio by weigh of human collagen type I to human collagen type III may be greater than about 50:50, or greater than about 70:30. The collagen of any embodiment herein may include a weight ratio of type I bovine collagen to type III bovine collagen of about 85:15.

In any embodiment described herein, the biomaterial may include from about 30 wt. % to about 70 wt. % of the thiolated collagen based on the total weight of the biomaterial. For example, in any embodiment disclosed herein, the amount of thiolated collagen may be about 38 wt. %, about 40 wt. %, about 42 wt. %, about 44 wt. %, about 46 wt. %, about 48 wt. %, about 50 wt. %, about 52 wt. %, about 56 wt. %, about 58 wt. %, about 60 wt. %, about 62 wt. %, about 64 wt. %, about 66 wt. %, about 68 wt. %, about 70 wt. %, or any range including and/or in between any two of these values based on the weight of the biomaterial, or any value including and/or in between any two of the preceding values based on the total weight of the biomaterial.

The collagen may have a weight-average molecular weight of about 5,000 to about 100,000. For example, in any embodiment disclosed herein, the collagen may have a weight-average molecular weight of about 5,000, about 6,000, about 7,000, about 8,000, about 9,000, about 10,000, about 12,000, about 14,000, about 16,000, about 18,000, about 20,000, about 22,000, about 24,000, about 26,000, about 28,000, about 30,000, about 32,000, about 34,000, about 36,000, about 38,000, about 40,000, about 45,000, about 50,000, about 55,000, about 60,000, about 65,000, about 70,000, about 75,000, about 80,000, about 85,000, about 90,000, about 95,000, about 100,000, or any range including and/or in between any two of the preceding values.

In any embodiment disclosed herein, the thiolated ORC may be an ORC thiolated as described herein. Oxidized regenerated cellulose (ORC) may be produced by the oxidation of cellulose, for example with dinitrogen tetroxide and/or as described in U.S. Pat. No. 3,122,478 (incorporated herein by reference). Not intending to be bound by theory, it is believed that this process may convert primary alcohol groups on the saccharide residues of the cellulose to carboxylic acid groups, for example, forming uronic acid residues within the cellulose chain. The oxidation may not proceed with complete selectivity, and as a result hydroxyl groups on carbons 2 and 3 of the saccharide residue may be converted to the keto form. These ketone units may introduce an alkali labile link, which at pH 7 or higher initiates the decomposition of the polymer via formation of a lactone and sugar ring cleavage. As a result, oxidized regenerated cellulose is biodegradable and bioresorbable under physiological conditions. ORC is available with a variety of degrees of oxidation and hence rates of degradation. The ORC may include particles, fibers, or both; in any embodiment disclosed herein, the ORC may be in the form of particles, such as fiber particles or powder particles. In embodiments that include ORC fibers, the ORC fibers may have a volume fraction such that at least 80% of the fibers have lengths in the range from about 5 μm to about 1000 μm, or from about 250 μm to about 450 μm.

In any embodiment disclosed herein, the thiolated biopolymer may be a thiolated cellulose. As discussed above, the biomaterial may include about 30 wt. % to about 70 wt. % of the thiolated ORC based on the total weight of the biomaterial. In any embodiment disclosed herein, the thiolated ORC may have a weight-average molecular weight of about 50,000 to about 1,000,000. Thus, the biomaterial may include the thiolated ORC of any embodiment disclosed herein in an amount of about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 56 wt. %, about 58 wt. %, about 60 wt. %, about 62 wt. %, about 64 wt. %, about 66 wt. %, about 68 wt. %, about 70 wt. %, or any range including and/or in between any two of these values based on the weight of the biomaterial. The thiolated ORC may have a weight-average molecular weight of about 50,000, about 55,000, about 60,000, about 65,000, about 70,000, about 75,000, about 80,000, about 85,000, about 90,000, about 95,000, about 100,000, about 110,000, about 120,000, about 130,000, about 140,000, about 150,000, about 160,000, about 170,000, about 180,000, about 190,000, about 200,000, about 210,000, about 220,000, about 230,000, about 240,000, about 250,000, about 260,000, about 270,000, about 280,000, about 290,000, about 300,000, about 310,000, about 320,000, about 330,000, about 340,000, about 350,000, about 360,000, about 370,000, about 380,000, about 390,000, about 400,000, about 410,000, about 420,000, about 430,000, about 440,000, about 450,000, about 460,000, about 470,000, about 480,000, about 490,000, about 500,000, about 510,000, about 520,000, about 530,000, about 540,000, about 550,000, about 560,000, about 570,000, about 580,000, about 590,000, about 600,000, about 610,000, about 620,000, about 630,000, about 640,000, about 650,000, about 660,000, about 670,000, about 680,000, about 690,000, about 700,000, about 710,000, about 720,000, about 730,000, about 740,000, about 750,000, about 760,000, about 770,000, about 780,000, about 790,000, about 800,000, about 810,000, about 820,000, about 830,000, about 840,000, about 850,000, about 860,000, about 870,000, about 880,000, about 890,000, about 900,000, about 910,000, about 920,000, about 930,000, about 940,000, about 950,000, about 960,000, about 970,000, about 980,000, about 990,000, about 1,000,000, or any range including and/or in between any two of the preceding values.

The thiolated ORC may include particles, fibers, or both; in any embodiment disclosed herein, the thiolated ORC may be in the form of particles, such as fiber particles or powder particles. In embodiments that include thiolated ORC fibers, the thiolated ORC fibers may have a volume fraction such that at least 80% of the fibers have lengths in the range from about 5 μm to about 1,000 μm. In any embodiment herein, the thiolated ORC may include fiber lengths of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 55 μm, about 60 μm, about 65 μm, about 70 μm, about 75 μm, about 80 μm, about 85 μm, about 90 μm, about 95 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 230 μm, about 240 μm, about 250 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1,000 μm, or any range including and/or in between any two of the preceding values.

In any embodiment disclosed herein, the biomaterial may include a weight ratio of thiolated collagen to thiolated ORC of about 70:30, about 69:31, about 68:32, about 67:33, about 66:34, about 65:35, about 64:34, about 63:33, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, or any range including and/or in between any two of these values. Additionally or alternatively, in some embodiments, the weight ratio of the thiolated collagen to thiolated ORC may be about 55:45.

The biomaterial includes one or more agents selected from growth factors, peptides, or combinations thereof, where the one or more agents may be conjugated to the thiolated biopolymer. For example, in any embodiment herein, the one or more agents may be conjugated to the thiolated biopolymer via a disulfide linkage.

In any embodiment disclosed herein, the growth factors may include one or more of fibroblast growth factors (FGFs), insulin-like growth factor (IGF), keratinocyte growth factor (KGF), vascular endothelial growth factor (VEGF), transforming growth factor β (TGFβ), epidermal growth factor (EGF), hepatocyte growth factor (HGF), or platelet-derived growth factor (PDGF). Additionally or alternatively, in some embodiments, the fibroblast growth factors comprise one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

In any embodiment disclosed herein, the peptides may include one or more of defensins, histatins, cathelicidin LL-37, or any combination of two or more thereof.

The one or more agents as disclosed herein in any embodiment may be present in the biomaterial in an amount from about 0.01 wt. % to about 20 wt. % of the one or more agents based on the total weight of the biomaterial. Suitable amounts of the one or more agents in the biomaterial may include, but are not limited to, about 0.01 wt. %, about 0.05 wt. %, about 0.1 wt. %, about 0.3 wt. %, about 0.5 wt. %, about 0.7 wt. %, about 0.9 wt. %, about 1 wt. %, about 2 wt. %, about 3 wt. %, about 4 wt. %, about 5 wt. %, about 6 wt. %, about 7 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, or any range including and/or in between any two of the preceding values based on the weight of the biomaterial.

In any embodiment disclosed herein, the biomaterial may further include one or more additional agents conjugated to the thiolated biopolymer. For example, in any embodiment disclosed herein, the one or more additional agents may include antimicrobial agents, antioxidants, silver compounds, or a combination of any two or more thereof. Exemplary antimicrobial agents may include, but are not limited to, polyhexamethylene biguanide, acetic acid, benzoic acid, povidine iodide, natamycin, nisin, citric acid, sorbic acid, propionic acid, honey sulfites, or a combination of two or more thereof. Exemplary antioxidants include, but are not limited to, anthocyanins, astaxanthin, bilirubin, canthaxanthin, capsaicin, curcumin, coenzyme Q10, eugenol, flavanol, flavonolignans, flavanone, flavone, flavonol, iodide, isoflavone phytoestrogen, lutein, lycopene, manganese, melatonin, N-acetylcysteine, oxalic acid, phenolic acid, phytic acid, R-α-lipoic acid, stilbenoid, tocopherol, tocotrienol, vitamin A, vitamin C, vitamin E, xanthones, zeaxanthin, α-carotene, β-carotene, as well as a combination of any two or more thereof.

In any embodiment disclosed herein, the biomaterial may further include any of the one or more agents as disclosed herein in addition to the one or more agents conjugated to the thiolated biopolymer. For example, in any embodiment disclosed herein, the biomaterial may include from about 0.01 wt. % to about 40.0 wt. % of the one or more non-conjugated agents based on the total weight of the biomaterial. Suitable amounts of the one or more non-conjugated agents may include, but are not limited to, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 15 wt. %, about 20 wt. %, about 25 wt. %, about 30 wt. %, about 35 wt. %, about 40 wt. %, or any range including and/or in between any two of these values based on the weight of the biomaterial.

In particular, in any embodiment disclosed herein, the biomaterial may further include a silver compound in addition to the one or more agents conjugated to the thiolated biopolymer. The biomaterial may include may include about 0.1 wt. % to about 3 wt. % of the silver compound. Thus, the silver compound may be included in the biomaterial in an amount of about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.22 wt. %, about 0.24 wt. %, about 0.26 wt. %, about 0.28 wt. %, about 0.3 wt. %, about 0.32 wt. %, about 0.34 wt. %, about 0.36 wt. %, about 0.38 wt. %, about 0.4 wt. %, about 0.42 wt. %, about 0.44 wt. %, about 0.46 wt. %, about 0.48 wt. %, about 0.50 wt. %, about 0.52 wt. %, about 0.54 wt. %, about 0.56 wt. %, about 0.58 wt. %, about 0.6 wt. %, about 0.62 wt. %, about 0.64 wt. %, about 0.66 wt. %, about 0.68 wt. %, about 0.7 wt. %, about 0.72 wt. %, about 0.74 wt. %, about 0.76 wt. %, about 0.78 wt. %, about 0.8 wt. %, about 0.82 wt. %, about 0.84 wt. %, about 0.86 wt. %, about 0.88 wt. %, about 0.9 wt. %, about 0.92 wt. %, about 0.94 wt. %, about 0.96 wt. %, about 0.98 wt. %, about 1 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, or any range including and/or in between any two of the preceding values.

In any embodiment disclosed herein, the silver compound may be one or more pharmaceutically acceptable salts. Additionally or alternatively, in some embodiments, the one or more pharmaceutically acceptable silver salts may include, but are not limited to, silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver ρ-aminobenzoate, silver ρ-aminosalicylate, nanocrystalline silver, any pharmaceutically acceptable salt thereof, a silver oxysalt (e.g., Ag₇NO₁₁), or a combination of any two or more thereof.

In any embodiment disclosed herein, the biomaterial may be in the form of a sponge, a film, a foam, a gel, a bead, a rope, a polymeric matrix, a coating, or a solution. In any embodiment disclosed herein, the biomaterial may be in the form of a foam.

Without being bound by theory, it is believed that the exposure of the biomaterial of the present technology to an acidic environment (e.g., an acidic wound environment) triggers release of the one or more agents (i.e., growth factors and/or peptides) conjugated to the thiolated biopolymer. The growth factors and/or peptides become free to assist with wound healing progression, and thus the functional properties of the biomaterial are tailorable and provide sustained release of the one or more agents. Exposure to an acidic wound environment (such as at pH of below 7) cleaves the disulfide bond linkage between the thiolated biopolymer and the one or more agents (i.e., growth factors and/or peptides), releasing the one or more agents into the wound. Additionally or alternatively, in some embodiments, the one or more agents (i.e., growth factors and/or peptides) may not be released under basic conditions (e.g., basic wound environment), and thus, the biomaterial may provide tailored and/or sustained release of the one or more agents conjugated to the thiolated biopolymer.

In any embodiment disclosed herein, the one or more agents that may include growth factors, peptides, or combinations thereof undergo cleavage from the thiolated biopolymer upon exposure to acidic conditions. For example, in any embodiment disclosed herein, the acidic conditions may include an acidic wound environment. Additionally or alternatively, in some embodiments, exposure to the acidic conditions or acidic wound environment may be induced via use of an acidic wound cleanser. In any embodiment disclosed herein, such acidic conditions or acidic wound environments may have a pH below 7. For example, in any embodiment disclosed herein, the acidic conditions may have a pH below about 6.5, below about 6, below about 5.5, below about 5, below about 4.5, below about 4, or any range including and/or in between any two of these values.

In any embodiment disclosed herein, the biomaterial of the present technology may be sterile. In any embodiment disclosed herein, the biomaterial of the present technology may be packaged in a microorganism-impermeable container.

### Methods of Making the Biomaterial of the Present Technology

In another related aspect, the present technology provides a method for preparing a biomaterial as disclosed herein in any embodiment. The method includes reacting a biopolymer with a thiolactone to obtain a thiolated biopolymer, reacting the thiolated biopolymer with one or more agents selected from growth factors, peptides, or combinations thereof to obtain the biomaterial, wherein the one or more agents are conjugated to the thiolated biopolymer via a disulfide linkage.

In any embodiment disclosed herein, the biopolymer may be a biopolymer as disclosed herein. For example, in any embodiment disclosed herein, the biopolymer may be collagen, oxidized cellulose, a polysaccharide, chitosan, gelatin, hyaluronic acid, elastin, fibronectin, or a combination of nay two or more thereof. In any embodiment disclosed herein, the biopolymer may include a collagen, an oxidized cellulose, or a mixture thereof.

The collagen may be a collagen material as described herein in any embodiment. For example, in any embodiment disclosed herein, the collagen may include mammalian collagen. For example, in any embodiment described herein, the mammalian collagen may include recombinant or naturally occurring bovine collagen, human collagen, or a combination thereof. In any embodiment herein, the collagen may be a naturally occurring collagen, including but not limited to, type I human collagen, type III human collagen, type I bovine collagen, type III bovine collagen, or a combination of any two or more thereof. In any embodiment herein, the collagen may be recombinant collagen, including but not limited to, type I human recombinant collagen, type III human recombinant collagen, type I bovine recombinant collagen, type III bovine recombinant collagen, or a combination of any two or more thereof.

The oxidized cellulose may be an oxidized regenerated cellulose (ORC) of any embodiment disclosed herein.

In any embodiment disclosed herein, the thiolactone may be a substituted or unsubstituted thiolactone with a primary amine group of the biopolymer, where the thiolactone may be a 3- to 12-member ring having 2 to 10 carbons. In any embodiment disclosed herein, the thiolactone may be 3- to 8-membered ring having 2 to 6 carbons. In any embodiment disclosed herein, the thiolactone may be represented by the following structure:

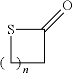

where n may be 0, 1, 2, 3, 4, 5, 6, 7, 8, or 9, and where the thiolactone is optionally substituted with an amide group. In any embodiment disclosed herein, n may be 2. In any embodiment disclosed herein, the thiolactone may be substituted with —NHC(O)CH₃. In any embodiment disclosed herein, the thiolactone may be N-acetyl homocysteine thiolactone.

In any embodiment disclosed herein, reacting the biopolymer with the thiolactone may include preparing a first mixture of the biopolymer and an aqueous bicarbonate buffer solution; combining the first mixture with the thiolactone and silver nitrate to obtain a second mixture, wherein the second mixture has a pH of about 6 to about 8; and contacting the biopolymer with the thiolactone in the presence of silver nitrate to obtain the thiolated biopolymer; wherein the reacting of the biopolymer occurs at a temperature of about 1° C. to about 25° C.

In any embodiment disclosed herein, the aqueous bicarbonate buffer solution may be a sodium bicarbonate solution. In any embodiment disclosed herein, the aqueous bicarbonate buffer solution may have a concentration of about 0.1 M to about 1 M solution. Suitable concentrations of the aqueous bicarbonate solution may include, but are not limited to, about 0.1 M, about 0.2 M, about 0.3 M, about 0.4 M, about 0.5 M, about 0.6 M, about 0.7 M, about 0.8 M, about 0.9 M, about 1M, or any range including and/or in between any two of these values.

In any embodiment disclosed herein, the first mixture may include the biopolymer at a concentration of about 0.1 mg/mL to about 20 mg/mL. For example, in any embodiment disclosed herein, the concentration of the biopolymer may be about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, or any range including and/or in between any two of these values.

In any embodiment disclosed herein, the second mixture may have a pH of about 6 to about 8, or about 7 to about 7.5. For example, in any embodiment disclosed herein, the second mixture may have a pH of about 6, about 6.5, about 7, about 7.5, about 8, or any range including and/or in between any two of these values.

In any embodiment disclosed herein, the second mixture may include a molar ratio of the thiolactone to the biopolymer of about 5:1 to about 35:1. For example, in any embodiment disclosed herein, the molar ratio may include, but is not limited to, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, or any range including and/or in between any two of these values.

In any embodiment disclosed herein, the temperature for reacting the biopolymer with the thiolactone may include, but is not limited to, about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., or any range including and/or in between any two of these values.

In any embodiment disclosed herein, reacting the biopolymer with the thiolactone may further include purifying the thiolated biopolymer; for example, in any embodiment disclosed herein, the purifying may include purification methods known in the art. Additionally or alternatively, in some embodiments, the thiolated biopolymer may be purified via dialysis.

In any embodiment disclosed herein, reacting the thiolated biopolymer with the one or more agents may occur under mild oxidizing conditions to form a disulfide linkage between the thiolated biopolymer and a thiol functionality of the one or more agents, and thus obtaining the biomaterial of the present technology. For example, in any embodiment disclosed herein, the mild oxidizing conditions may include reacting the thiolated biopolymer with the one or more agents (e.g., growth factors and/or peptides) in the presence of a mild oxidizing agent. Exemplary mild oxidizing agents may include, but are not limited to, iodine, glutathione, N-ethylmaleimide, ρ-chloromercuribenzoate, or combinations of two or more thereof.

In any embodiment disclosed herein, the growth factors may include one or more of fibroblast growth factors (FGFs), insulin-like growth factor (IGF), keratinocyte growth factor (KGF), vascular endothelial growth factor (VEGF), transforming growth factor β (TGFβ), epidermal growth factor (EGF), hepatocyte growth factor (HGF), or platelet-derived growth factor (PDGF). Additionally or alternatively, in some embodiments, the fibroblast growth factors comprise one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

In any embodiment disclosed herein, the peptides may include one or more of defensins, histatins, cathelicidin LL-37, or any combination of two or more thereof.

The one or more agents as disclosed herein in any embodiment may be present in the biomaterial in an amount from about 0.001 wt. % to about 20.0 wt. % of the one or more agents based on the total weight of the biomaterial. Suitable amounts of the one or more agents in the biomaterial may include, but are not limited to, about 0.01 wt. %, about 0.05 wt. %, about 0.1 wt. %, about 0.3 wt. %, about 0.5 wt. %, about 0.7 wt. %, about 0.9 wt. %, about 1 wt. %, about 2 wt. %, about 3 wt. %, about 4 wt. %, about 5 wt. %, about 6 wt. %, about 7 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, or any range including and/or in between any two of the preceding values based on the weight of the biomaterial.

In any embodiment disclosed herein, the biomaterial may further reacting the thiolated biopolymer with one or more additional agents. For example, in any embodiment disclosed herein, the one or more additional agents may include antimicrobial agents, antioxidants, or combinations thereof. Exemplary antimicrobial agents may include, but are not limited to, polyhexamethylene biguanide, acetic acid, benzoic acid, povidine iodide, natamycin, nisin, citric acid, sorbic acid, propionic acid, honey sulfites, or a combination of two or more thereof. Exemplary antioxidants include, but are not limited to, anthocyanins, astaxanthin, bilirubin, canthaxanthin, capsaicin, curcumin, coenzyme Q10, eugenol, flavanol, flavonolignans, flavanone, flavone, flavonol, iodide, isoflavone phytoestrogen, lutein, lycopene, manganese, melatonin, N-acetylcysteine, oxalic acid, phenolic acid, phytic acid, R-α-lipoic acid, stilbenoid, tocopherol, tocotrienol, vitamin A, vitamin C, vitamin E, xanthones, zeaxanthin, α-carotene, β-carotene, as well as a combination of any two or more thereof.

Representative Biomaterial Preparation Schemes

The illustrative biomaterial may be prepared as indicated in the following reaction schemes using procedures known to those of ordinary skill in the art.

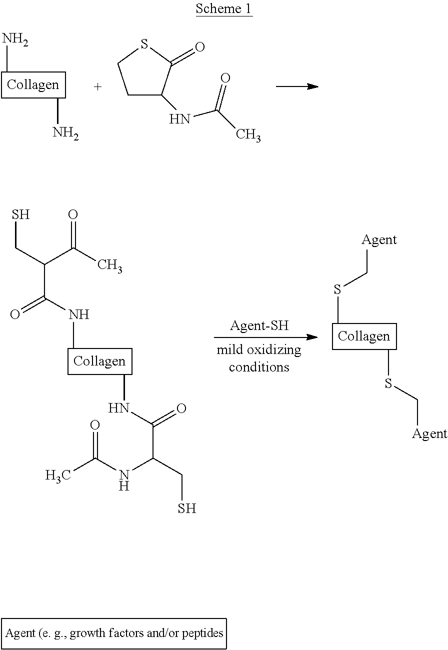

In any embodiment disclosed herein, the biomaterial may be formed into a sponge, a film, a foam, a gel, a bead, a rope, a polymeric matrix, a coating, or a solution. Additionally or alternatively, in some embodiments, the biomaterial may be in the form of a freeze dried sponge or a film material. In any embodiment disclosed herein, a suitable sponge is made by freeze drying or solvent drying an aqueous dispersion that includes the biomaterial of the present technology.

Wound Dressing and Treatment methods of the Present Technology

In a related aspect, the present technology provides a wound dressing that includes the biomaterial as disclosed herein in any embodiment.

In any embodiment disclosed herein, the wound dressing may be in the form of a sponge, a film, a foam, a gel, a bead, a rope, a polymeric matrix, a coating, or a solution. In any embodiment disclosed herein, the biomaterial may be in the form of a foam.

In any embodiment disclosed herein, the biomaterial of the present technology may be sterile. In any embodiment disclosed herein, the biomaterial of the present technology may be packaged in a microorganism-impermeable container.

In any embodiment disclosed herein, the wound dressing of the present technology may be sterile and packaged in a microorganism-impermeable container.

### Negative-Pressure Therapy

The biomaterial and/or wound dressing of any embodiment described herein may be employed in therapy in which a wound is treated with reduced pressure. Treatment of tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and/or micro-deformation of tissue at a wound site. Together, these benefits may increase development of granulation tissue and reduce healing times.

Generally, the system may be configured to provide negative-pressure to a wound in accordance with this specification. In any embodiment herein, the system may generally include a negative-pressure supply, and may include or be configured to be coupled to a distribution component. In general, a distribution component may refer to any complementary or ancillary component configured to be fluidly coupled to a negative-pressure supply in a fluid path between a negative-pressure supply and a wound.

In any embodiment herein, the biomaterial and/or wound dressing may be configured to distribute negative pressure. The biomaterial and/or wound dressing may comprise or be configured as a manifold. A “manifold” in this context generally includes any composition or structure providing a plurality of pathways configured to collect or distribute fluid across a wound under pressure. For example, a manifold may be configured to receive negative pressure from the negative-pressure source and to distribute negative pressure through multiple apertures (e.g., pores), which may have the effect of collecting fluid and drawing the fluid toward the negative-pressure source. Additionally or alternatively, the fluid path(s) may be reversed or a secondary fluid path may be provided to facilitate movement of fluid across a wound. Additionally or alternatively, the fluid pathways of a manifold may be interconnected to improve distribution or collection of fluids. Additionally or alternatively, a manifold may be a porous material having a plurality of interconnected cells or pores. For example, in any embodiment herein, open-cell foams such as reticulated foams may generally include pores, edges, and/or walls that may form interconnected fluid pathways (such as channels).

The fluid mechanics associated with using a negative-pressure source to reduce pressure in another component or location, such as within a sealed therapeutic environment, can be mathematically complex. However, the basic principles of fluid mechanics applicable to negative-pressure therapy are generally well-known to those skilled in the art. The process of reducing pressure may be described generally and illustratively herein as “delivering,” “distributing,” or “generating” negative pressure, for example.

In general, a fluid, such as wound fluid (for example, wound exudates and other fluids), flows toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” may generally refer to a pressure less than a local ambient pressure, such as the ambient pressure in a local environment external to a sealed therapeutic environment provided by the biomaterial and/or wound dressing. In many cases, the local ambient pressure may also be the atmospheric pressure proximate to or about a wound. Alternatively or additionally, the pressure may be less than a hydrostatic pressure associated with the tissue at the wound. While the amount and nature of negative pressure applied to a wound may vary according to therapeutic requirements, the pressure is generally a low vacuum, also commonly referred to as a rough vacuum, between −5 mm Hg (−667 Pa) and −500 mm Hg (−66.7 kPa), gauge pressure. Common therapeutic ranges are between −50 mm Hg (−6.7 kPa) and −300 mm Hg (−39.9 kPa), gauge pressure.

Additionally or alternatively, in any embodiment herein, a negative-pressure supply may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. A negative-pressure source may be combined with a controller and other components into a therapy unit. A negative-pressure supply may also have one or more supply ports configured to facilitate coupling and de-coupling of the negative-pressure supply to one or more distribution components.

In any embodiment herein, components may be fluidly coupled to each other to provide a path for transferring fluids (i.e., liquid and/or gas) between the components. For example, components may be fluidly coupled through a fluid conductor, such as a tube. As used herein, the term “fluid conductor” may include a tube, pipe, hose, conduit, or other structure with one or more lumina or open passages adapted to convey a fluid between two ends thereof. Typically, a fluid conductor may be an elongated, cylindrical structure with some flexibility, but the geometry and rigidity may vary. Additionally or alternatively, in any embodiment herein, the negative-pressure source may be operatively coupled to the dressing via a dressing interface.

### Treatment Methods

In an aspect, methods for treating a wound in a subject in need thereof are provided, wherein the method includes administering to the wound a wound dressing that includes the biomaterial of any embodiment disclosed herein. The wound may be a dermal wound, a diabetic wound, an acute wound, a chronic wound, or a combination of any two or more thereof. Exemplary acute wounds may include, but are not limited to, surgical wounds, trauma wounds, burn wounds, and/or donor sites. Exemplary chronic wounds include, but are not limited to, infectious wounds, venous ulcers, pressure ulcers, decubitis ulcers, diabetic ulcers, and/or stalled, non-healing wounds.

As previously described herein, the biomaterial of the wound dressing may be tailored to provide sustained release of the one or more agents that include growth factors, peptides, or combinations thereof, such that the wound dressings may release the one or more agents upon exposure to acidic conditions (e.g., an acidic wound environment). The thiolated biopolymer, being functionalized with the one or more agents as described herein in any embodiment, may be strategically released to promote and/or improve healing in a wound upon exposure to acidic conditions. As illustrated in the reaction scheme below, the biomaterial releases the one or more agents conjugated to the thiolated biopolymer following exposure to acidic conditions.

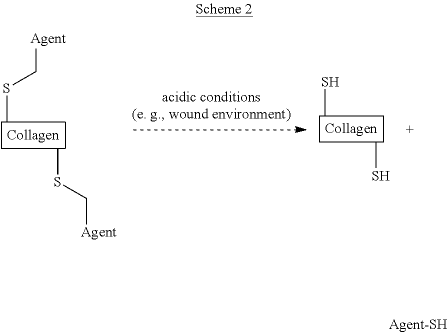

In any embodiment disclosed herein, the treatment may further include exposing the wound dressing to acidic conditions, to release (i.e., cleave) the one or more agents as disclosed herein from the thiolated biopolymer into the wound. For example, in any embodiment disclosed herein, the acidic conditions may include an acidic wound environment. Additionally or alternatively, in some embodiments, exposure to the acidic conditions or acidic wound environment may be induced via use of an acidic wound cleanser. In any embodiment disclosed herein, such acidic conditions or acidic wound environments may have a pH below 7. For example, in any embodiment disclosed herein, the acidic conditions may include having a pH below about 6.5, below about 6, below about 5.5, below about 5, below about 4.5, below about 4, or any range including and/or in between any two of these values. In any embodiment disclosed herein, the one or more agents cleaved from the thiolated biopolymer upon exposure to acidic conditions may include growth factors, peptides, biopolymers, or combinations of any two or more thereof. In any embodiment disclosed herein, one or more growth factors and/or peptides may be released upon exposure to acidic conditions. For example, in any embodiment disclosed herein, the growth factors and/or peptides may be released in a wound to accelerate wound healing.

In any embodiment disclosed herein, the one or more additional agents conjugated to the thiolated biopolymer, such as antimicrobial agents, may be released upon exposure to acidic conditions into the wound. For example, in any embodiment disclosed herein, the one or more additional agents may be antimicrobial agents as disclosed herein; for example, the one or more antimicrobial agents may be released in a wound having a risk of infection and/or an infected wound. The infection may be a bacterial infection or a fungal infection. The bacterial infection may be caused by gram-negative or gram-positive bacteria.

Examples of gram-positive bacteria include, but are not limited to, Actinomyces sp., Arcanobacterium sp., Bacillus sp., Bavariicoccus sp., Brachybacterium sp., Clostridium sp., Cnuibacter sp., Corynebacterium sp., Enterococcus sp., Desulfitobacterium sp., Fervidobacterium sp., Georgenia sp., Janibacter sp., Lactobacillales sp., Microbispora sp., Nocardia sp., Pasteuria sp., Pilibacter sp., Propionibacterium sp., Rathayibacter sp., Rhodococcus sp., Roseburia sp., Rothia sp., Sarcina sp., Solibacillus sp., Sporosarcina sp., Staphylococcus sp., Streptococcus sp., Syntrophomonas sp., or Tepidibacter sp.

Examples of gram-negative bacteria include, but are not limited to, Acetobacter sp., Acidaminococcus sp., Acinetobacter sp., Agrobacterium sp., Akkermansia sp., Anaerobiospirillum sp., Anaerolinea sp., Arcobacter sp., Armatimonas sp., Azotobacter sp., Bacteroides sp., Bacteroidetes sp., Bartonella sp., Bdellovibrio sp., Brachyspira sp., Bradyrhizobium sp., Caldilinea sp., Cardiobacterium sp., Christensenella sp., Chthonomonas sp., Coxiella sp., Cyanobacteria sp., Cytophaga sp., Dehalogenimonas sp., Desulfurobacterium sp., Devosia sp., Dialister sp., Diciyoglomus sp., Dinoroseobacter sp., Enterobacter sp., Escherichia sp., Fimbriimonas sp., Flavobacterium sp., Francisella sp., Fusobacterium sp., Gluconacetobacter sp., Haemophilus sp., Helicobacter sp., Kingella sp., Klebsiella sp., Kluyvera sp., Kozakia sp., Legionella sp. Leptonema sp. Leptotrichia sp., Levilinea sp. Luteimonas sp. Megamonas sp., Megasphaera sp., Meiothermus sp., Methylobacterium sp., Moraxella sp., Morganella sp., Mycoplasma sp., Neisseria sp., Nitrosomonas sp., Pectinatus sp., Pedobacter sp., Pelosinus sp., Propionispora sp., Proteus sp., Pseudomonas sp., Pseudoxanthomonas sp., Rickettsia sp., Salinibacter sp., Salmonella sp., Samsonia sp., Serratia sp., Shigella sp., Shimwellia sp., Sphingomonas sp., Stenotrophomonas sp., Thorselliaceae sp., Vampirococcus sp., Verminephrobacter sp., Vibrio sp., Victivallis sp., Vitreoscilla sp., Wolbachia sp.

In any embodiment disclosed herein, the infection may be caused by Aspergillus sp., Aureobasidium sp., Candida sp., Cladosporium sp., Curvularia sp., Engodontium sp., Epicoccum sp., Gibberella sp., Hypocreales sp., Leptosphaerulina sp., Malessezia sp., Penicillium sp., Rhodosporidium sp., Trichosporon sp., Trichtophyton sp., and Ulocladium sp.

Additionally or alternatively, in some embodiments, the wound dressing is administered directly to the wound. Any method known to those in the art for administering a wound dressing to an acute or a chronic wound disclosed herein may be employed. Suitable methods include in vitro or in vivo methods. In vivo methods typically include the administration of one or more wound dressings to a subject in need thereof, suitably a human. When used in vivo for therapy, the one or more wound dressings described herein are administered to the subject in effective amounts (i.e., amounts that have desired therapeutic effect). The dose and dosage regimen will depend upon the state of the wound of the subject, and the characteristics of the particular wound dressing used.

The effective amount may be determined during pre-clinical trials and clinical trials by methods familiar to physicians and clinicians. An effective amount of one or more wound dressings useful in the methods may be administered to a subject in need thereof by any number of well-known methods for administering wound dressings.

In any embodiment disclosed herein, the wound dressings may be administered daily for 1 hour or more, for 2 hours or more, for 3 hours or more, for 4 hours or more, for 5 hours or more, for 6 hours or more, for 12 hours or more. In any embodiment disclosed herein, the wound dressings may be administered one, two, three, four, or five times per day. In any embodiment disclosed herein, the wound dressings may be administered daily for one, two, three, four or five weeks. In any embodiment disclosed herein, the wound dressings may be administered daily for less than 6 weeks. In any embodiment disclosed herein, the wound dressings may be administered daily for 6 weeks or more. In any embodiment disclosed herein, the wound dressings may be administered daily for 12 weeks or more. In any embodiment disclosed herein, the wound dressings may be administered every day, every other day, every third day, every fourth day, every fifth day, or every sixth day. In any embodiment disclosed herein, the wound dressings may be administered weekly, bi-weekly, tri-weekly, or monthly. In any embodiment disclosed herein, the wound dressings may be administered for a period of one, two, three, four, or five weeks. In any embodiment disclosed herein, the wound dressings may be administered for six weeks or more. In any embodiment disclosed herein, the wound dressings may be administered for twelve weeks or more. In any embodiment disclosed herein, the wound dressings may be administered for a period of less than one year. In any embodiment disclosed herein, the wound dressings may be administered for a period of more than one year. In any embodiment disclosed herein, the wound dressings may be administered for a chronic wound as appropriate.

In any embodiment herein, the process may further include employing the wound dressing in the context of a negative-pressure therapy, where the negative-pressure therapy may include positioning the wound dressing proximate to the wound. For example, the various components of the dressing may be positioned with respect to the wound sequentially or, alternatively, may be positioned with respect to each other and then positioned with respect to the wound. The negative-pressure therapy may further comprise sealing the wound dressing to tissue surrounding the wound to form a sealed space. For example, the wound dressing may be positioned proximate to the wound and sealed to an attachment surface near the wound, for example, to undamaged epidermis peripheral to a wound.

The negative-pressure therapy method in any embodiment herein may further include fluidly coupling a negative-pressure source to the sealed space and operating the negative-pressure source to generate a negative pressure in the sealed space. For example, the negative-pressure source may be coupled to the dressing such that the negative-pressure source may be used to reduce the pressure in the sealed space. For example, negative pressure applied across the wound, for example, via the dressing may be effective to induce macrostrain and microstrain at the wound, as well as remove exudates and other fluids from the wound.

### Kits Comprising the Wound Dressing of the Present Technology

In a further related aspect, the present disclosure provides kits that include a wound dressing that includes the biomaterial as disclosed herein in any embodiment and instructions for use. The kits of the present technology may also include methods for treating a wound in a subject in need thereof. The kit may optionally comprise components such as antiseptic wipes, ointment, adhesive tape, tweezers, scissors, etc.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A biomaterial comprising a thiolated biopolymer and one or more agents comprising growth factors, peptides, or combinations thereof; wherein the one or more agents are conjugated to the thiolated biopolymer.

2. The biomaterial of claim 1, wherein the thiolated biopolymer comprises from about 0.01 weight percent (wt. %) to about 99 wt. % of the biomaterial based on the weight of the biomaterial.

3. The biomaterial of claim 1, wherein the thiolated biopolymer is thiolated by reaction of a thiolactone with a primary amine group of a biopolymer, wherein the thiolactone is a 3- to 12-membered ring comprising 2 to 10 carbons.

4. The biomaterial of claim 1, wherein the thiolated biopolymer comprises thiolated collagen, thiolated cellulose, or a mixture thereof.

5.-8. (canceled)

9. The biomaterial of claim 1, wherein the thiolated biopolymer comprises thiolated collagen, the thiolated collagen has a weight-average molecular weight of about 5,000 to about 100,000, and wherein the thiolated collagen comprises about 30 wt. % to about 70 wt. % of the biomaterial based on the total weight of the biomaterial.

10. The biomaterial of claim 1, wherein the thiolated biopolymer comprises thiolated oxidized regenerated cellulose (ORC), the thiolated ORC has a weight-average molecular weight of about 50,000 to about 1,000,000, and wherein the thiolated ORC comprises about 30 wt. % to about 70 wt. % of the biomaterial based on the total weight of the biomaterial.

11.-12. (canceled)

13. The biomaterial of claim 1, wherein the one or more agents comprise about 0.01 wt. % to about 20 wt. % of the biomaterial based on the total weight of the biomaterial.

14. The biomaterial of claim 13, wherein the one or more agents comprise one or more growth factors conjugated to the thiolated biopolymer, and wherein the one or more growth factors comprise fibroblast growth factors (FGFs), insulin-like growth factor (IGF), keratinocyte growth factor (KGF), vascular endothelial growth factor (VEGF), transforming growth factor β (TGFβ), epidermal growth factor (EGF), hepatocyte growth factor (HGF), platelet-derived growth factor (PDGF), or combinations of two or more thereof.

15. The biomaterial of claim 13, wherein the one or more agents comprise one or more peptides conjugated to the thiolated biopolymer, and wherein the one or more peptides comprise defensins, histatins, cathelicidin LL-37, or combinations of two or more thereof.

16. The biomaterial of claim 1, wherein the one or more agents are conjugated to the thiolated biopolymer via a disulfide linkage.

17. The biomaterial of claim 1, wherein the biomaterial further comprises one or more additional agents conjugated to the thiolated biopolymer, and wherein the one or more additional agents are selected from antimicrobial agents, antioxidants, or combinations thereof.

18.-19. (canceled)

20. The biomaterial of claim 1, wherein the biomaterial is in the form of a foam or a sponge.

21. The biomaterial of claim 1, wherein the one or more agents undergo cleavage from the biomaterial thiolated biopolymer in acidic conditions.

22. (canceled)

23. A method for treating a wound in a subject in need thereof, the method comprising:
administering directly to the wound a wound dressing comprising the biomaterial of claim 1; and
exposing the wound dressing to acidic conditions to cleave the one or more agents from the thiolated biopolymer of the biomaterial in the wound dressing into the wound

24. (canceled)

25. The method of claim 23, further comprising administering negative pressure to the wound subsequent to administering the wound dressing to the wound.

26. (canceled)

27. A method for preparing a biomaterial, the method comprising:
reacting a biopolymer with a thiolactone to obtain a thiolated biopolymer;
reacting the thiolated biopolymer with one or more agents selected from growth factors, peptides, or combinations thereof to obtain the biomaterial;
wherein reacting the thiolated biopolymer with the one or more agents is performed under mild oxidizing conditions such that the one or more agents are conjugated to the thiolated biopolymer via a disulfide linkage between the thiolated biopolymer and a thiol functionality of the one or more agents.

28. The method of claim 27, wherein reacting the biopolymer with the thiolactone comprises:
preparing a first mixture of the biopolymer and a bicarbonate buffer solution;
combining the first mixture with the thiolactone and silver nitrate to obtain a second mixture, wherein the second mixture comprises a molar ratio of the thiolactone to the biopolymer of about 5:1 to about 35:1, and wherein the second mixture has a pH of about 6 to about 8; and
contacting the biopolymer with the thiolactone in the presence of silver nitrate to obtain the thiolated biopolymer;
wherein the reacting of the biopolymer occurs at a temperature of about 1° C. to about 25° C.

29.-33. (canceled)

34. The method of claim 27, wherein the mild oxidizing conditions comprise reacting the thiolated biopolymer with the one or more agents in the presence of a mild oxidizing agent comprising iodine, glutathione, N-ethyhnaleimide, ρ-chloromercuribenzoate, or a combination of two or more thereof.

35.-40. (canceled)

41. The method of claim 27, wherein the thiolactone is N-acetyl homocysteine thiolactone.

42. The method of claim 27, comprising reacting the thiolated biopolymer with one or more growth factors and one or more peptides, the one or more growth factors comprising fibroblast growth factors (FGFs), insulin-like growth factor (IGF), keratinocyte growth factor (KGF), vascular endothelial growth factor (VEGF), transforming growth factor β (TGFβ), epidermal growth factor (EGF), hepatocyte growth factor (HGF), platelet-derived growth factor (PDGF), or combinations of two or more thereof, and the one or more peptides comprising defensins, histatins, cathelicidin LL-37, or combinations of two or more thereof.

43.-44. (canceled)
